(19) United States
(12) Patent Application Publication
(10) Pub. No.: US 2005/0136405 A1
(43) Pub. Date: Jun. 23, 2005

(54) Novel method for the detection of cancer biomarkers in cervical specimens

(76) Inventors: James Linder, Acton, MA (US); Brian Lentrichia, Acton, MA (US)

Correspondence Address: Theodore Allen, Esq.;Cytyc Corporation, 85 Swanson Road, Boxborough, MA, 01719, US

(21) Appl. No.: 10/738,018

(22) Filed: Dec. 17, 2003

(51) Int. Cl. C12Q 1/68; G01N 33/574

(52) U.S. Cl. 435/6; 435/7.23

(57) ABSTRACT

The invention provides a method for the assessment of biological markers in a sample of tissue, cells or fluid for the detection a malignant or pre-malignant condition. Aspects of the present invention are particularly useful in screening samples such as cervical smears from women to detect such markers. Identification of a malignant or pre-malignant condition may be followed by appropriate treatment.

### BACKGROUND OF THE INVENTION

Cancer is responsible for significant health problems in populations of women in the United States and throughout the world. In particular, gynecological cancers, including ovarian, uterine, cervical, and vuvlar cancers, are responsible for over 5,000 deaths in the United States each year. Although recent years have seen advances in detection and treatment of these cancers, mortality rates remain significantly high.

Despite being the third most prevalent gynecological cancer, ovarian cancer is the leading cause of death among those afflicted with gynecological cancers. The disproportionate mortality of ovarian cancer is attributable to a substantial absence of symptoms among those afflicted with early-stage ovarian cancer and to difficulty diagnosing ovarian cancer at an early stage. Patients afflicted with ovarian cancer most often present with non-specific complaints, such as abnormal vaginal bleeding, gastrointestinal symptoms, urinary tract symptoms, lower abdominal pain, and generalized abdominal distension. These patients rarely present with paraneoplastic symptoms or with symptoms that clearly indicate their affliction. Presently, less than about 40% of patients afflicted with ovarian cancer present with stage I or stage II. Management of ovarian cancer would be significantly enhanced if the disease could be detected at an earlier stage, when treatments are much more generally efficacious.

Tumors of the uterus are the most common group of gynecological cancers. Over 35,000 cases of uterine cancer are diagnosed in the United States every year. If discovered early, this slow-growing form of cancer is likely to be confined to the uterus, however, there are no useful screening tests and routine pelvic exams rarely detect this disease. Uterine cancer can be detected at an early stage due to vaginal bleeding between menstrual cycles or after menopause. The 5-year survival rate of patients diagnosed with either Stage I or Stage II uterine cancer is 90% and 75% respectively. Management of uterine cancer would be significantly enhanced if the disease could be detected at an earlier stage, when treatments are much more generally efficacious. The majority of endometrial cancer cases present with post-menopausal bleeding and are typically confirmed by histology of biopsy specimens collected by the Pipelle device (manufacturer?). Surgical intervention (i.e. hysterectomy) is curative in these patients. Some cases however may present in pre-menopausal women when hysterectomy is not as viable an option. In pre-menopausal cases, early detection may allow the option of a more localized treatment without the need for radical hysterectomy that would result in infertility. Another advantage of biomarkers for detection of endometrial cancer and its precursors is that it would reduce the proportion costly and time-consuming biopsy procedures performed. For example, only in an instance when a biomarker(s) is present, would a biopsy specimen be obtained for confirmation.

Endometriosis is a female disease in which endometrial tissue is found outside the uterus, its normal anatomic location, and it affects women of reproductive age, causing substantial debilitation and possible sterility or infertility, depending upon the severity of the condition. Endometrial tissue is improperly implanted in other anatomical sites, such as the peritoneal cavity, kidneys, and more often the ovaries. See Taylor, et al., Brit. J. Ob & Gyn., 98:680-684 (1991); and Vigano, et al., Fertility and Sterility, 56:894 (1991); Badaway, et al., Fertility and Sterility, 53:930 (1990). Endometrial cancer occurs at a rate of approximately 44,500 new cases per year with approximately 10,000 deaths per year. If diagnosed and treated early, when the cancer is still confined to the endometrium, cure can be achieved in approximately 95% of the cases by hysterectomy. Pap smears can show endometrial cancers but are effective in only 50% of the cases.

Clinical signs and symptoms usually consist of severe dysmenorrhea, dyspareunia and pelvic pain due to intrapelvic bleeding and periuterine adhesions. Nodules with a red-blue to yellow-brown appearance are found on or just beneath the surfaces of the site of involvement. Extensive fibrous adhesions can be found among the reproductive structures, such as the ovaries. The disease is histologically diagnosed if two of the three following features are identified outside the uterine cavity: endometrial glands, stroma and hemosiderin pigment. See Robbins, Pathologic Basis of Disease 5th Edition, W. B. Saunders Company, Philadelphia (1994).

Currently, laparoscopy is the procedure of choice for the diagnosis of endometriosis, because it enables the surgeon to possibly evaluate the extent of the disease. Other modalities for evaluation of suspected endometriosis include measurement of serum cancer antigen 125 (CA 125) and imaging studies, such as ultrasound and magnetic resonance imaging. However, these diagnostic tools have their limitations, since they do not allow for distinguishing endometriosis over other physiological situations, such as benign pelvic or ovarian conditions. Management of gynecological cancers currently relies on a combination of early diagnosis and aggressive treatment, which may include one or more of a variety of treatments such as surgery, radiotherapy, chemotherapy and hormone therapy. The course of treatment for a particular cancer is often selected based on a variety of prognostic parameters, including an analysis of specific tumor biomarkers. However, the use of established biomarkers often leads to a result that is difficult to interpret, and high mortality continues to be observed in many cancer patients.

The present invention relates to assessment of biological markers in a sample of tissue, cells or fluid with a view to detecting a malignant or pre-malignant condition. Aspects of the present invention are particularly useful in screening samples such as cervical smears from women to detect such markers. Identification of a malignant or pre-malignant condition may be followed by appropriate treatment following more extensive diagnostic procedures.

Accordingly, there is a need in the art for improved methods for detecting and treating cancers such as ovarian and endometrial cancer. The present invention fulfills these needs and further provides other related advantages.

### SUMMARY OF THE INVENTION

The present invention allows for the detection of markers in a biological sample, comprising the steps of obtaining a cervical pap specimen and detecting the presence of markers in the sample wherein the presence of the markers is indicative of the presence of cancer.

In one embodiment of the present invention, said cancer is ovarian or endometrial cancer. In another embodiment of the present invention, the markers are freely soluble or membrane associated. In another embodiment of the present invention, the markers are selected from the group consisting of proteins, nucleic acids, carbohydrates, fatty acids, glycoproteins, and lipids. In another embodiment of the present invention, the sample collected is from a cervical scraping collected for liquid-based cytology.

In a preferred embodiment, the invention allows for detecting ovarian cancer markers in a pap smear, comprising the steps of obtaining a cervical pap specimen and detecting the presence of markers in the sample wherein the presence of markers is indicative of ovarian cancer.

In a preferred embodiment, the invention allows for detecting endometrial cancer markers in a pap smear, comprising the steps of obtaining a cervical pap specimen and detecting the presence of markers in the sample wherein the presence of markers is indicative of endometrial cancer.

Other and further aspects, features, and advantages of the present invention will be apparent from the following description of the presently preferred embodiments of the invention. These embodiments are given for the purpose of disclosure.

Definitions

As used herein, the term “agent” refers to anything that cancer cells, including tumor cells, may be exposed to in a therapeutic protocol. In the context of the present invention, such agents include, but are not limited to, chemotherapeutic agents, such as anti-metabolic agents, e.g., Ara AC, 5-FU and methotrexate, antimitotic agents, e.g., TAXOL, inblastine and vincristine, alkylating agents, e.g., melphanlan, BCNU and nitrogen mustard, Topoisomerase II inhibitors, e.g., VW-26, topotecan and Bleomycin, strand-breaking agents, e.g., doxorubicin and DHAD, cross-linking agents, e.g., cisplatin and CBDCA, radiation and ultraviolet light. In a preferred embodiment, the agent is a taxane compound (e.g., TAXOL) and/or a platinum compound (e.g., cisplatin).

As used herein, the term “biological sample” refers to any tissue or material derived from a living or dead human which may contain the markers, including, for example, peripheral blood or bone marrow, plasma, serum, cervical swab samples, biopsy tissue including lymph nodes, respiratory tissue or exudates, gastrointestinal tissue, urine, feces, semen or other body fluids, tissues or materials. The biological sample may be treated to physically or mechanically disrupt tissue or cell structure, thus releasing intracellular components into a solution which may contain enzymes, buffers, salts, detergents and the like which are used to prepare the biological sample using standard methods for analysis. In a preferred embodiment, the biological sample is a cervical scraping collected for liquid-based cytology.

As used herein, the term “cancer cells”, including tumor cells, refer to cells that divide at an abnormal (increased) rate. Cancer cells include, but are not limited to, carcinomas, such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; leukemias and lymphomas such as granulocytic leukemia, monocytic leukemia, lymphocytic leukemia, malignant lymphoma, plasmocytoma, reticulum cell sarcoma, or Hodgkins disease; and tumors of the nervous system including glioma, meningoma, medulloblastoma, schwannoma or epidymoma.

As used herein, the term “marker” or “biomarker” refers to without limitation, organic as well as inorganic substances consisting of lipids, lipopolysaccharides, fatty acids, carbohydrates, sugars, proteins, glycoproteins, endogenous enzymes such as kinases, cell membrane structures, cytoplasmic, nucleotides and nucleic acids (i.e., DNA, RNA). The markers particularly include any markers of diagnostic interest and especially proliferation markers such as; hormone receptors such as estrogen receptors, progesterone receptors or androgen receptors; cytoskeleton compounds; hematological markers; oncogene products such as p53; cell membrane constituents; nuclear bound receptors; chromosomal aberrations such as gene amplifications, gene deletions, point mutations and translocations; and infectious agents.

As used herein, the term “ovarian cancer” refers to, but is not limited to ovarian tumors, carcinomas, (e.g., carcinoma in situ, invasive carcinoma, metastatic carcinoma) and pre-malignant conditions. By “ovarian tumor” is meant both benign and malignant tumors, such as ovarian germ cell tumors, e.g. teratomas, dysgerminoma, endodermal sinus tumor and embryonal carcinoma, and ovarian stromal tumors, e.g. granulosa, theca, Sertoli, Leydig, and collagen-producing stromal cells. Ovarian cancers as used herein also include art recognized histological tumor types, which include, for example, serous, mucinous, endometrioid, and clear cell tumors. The term ovarian cancer as used herein further includes art recognized grade and stage scales: grade I, II and III and stage I (including stage IA, IB and IC), II (including stage IIA, IIB and IIC), III (including stage IIIA, IIIB and IIIC), and IV.

As used herein, the term “endometrial cancer” refers to, but is not limited to endometrial carcinomas and endometrial adenocarcinomas. Endometrial cancers as used herein also include other well-known cell types such as papillary serous carcinoma, clear cell carcinoma, papillary endometrioid carcinoma, and mucinous carcinoma.

This invention is further illustrated by the following examples that should not be construed as limiting. The contents of all references, patents and published patent applications cited throughout this application are incorporated herein by reference.

Other terms used in the fields of molecular and cell biology and the DNA recombination as used herein should be generally understood well by the person of ordinary skill in the applicable arts.

### DETAILED DESCRIPTION OF THE INVENTION

The lining of the female reproductive tract is contiguous between the cervix and the ovaries via the fallopian tubes and the uterus. As such, soluble or non-membrane associated markers can be transported to the cervical canal by the mucin coating and actuated cilia in much the same way that a mature oocyte is transported to the uterine lining. The presence of abnormal cell grow within the reproductive tract (i.e., cancers) would change the composition and/or amount of the markers present in the mucin lining.

Accordingly, the present invention describes a method for detecting markers in a biological sample, comprising the steps of: (a) obtaining a cervical pap specimen and (b) detecting the presence of markers in said sample, wherein the presence of said markers is indicative of cancer. In a preferred embodiment, the cancer is ovarian or endometrial cancer. In another preferred embodiment, the markers are selected from the group consisting of proteins, nucleic acids, carbohydrates, fatty acids, glycoproteins, and lipids. In yet another embodiment, the markers are soluble proteins.

The present invention provides methods for detecting ovarian cancer markers in a pap smear comprising the steps of: (a) obtaining a cervical pap smear, and (b) detecting the presence of said markers in said sample, wherein the presence of said markers is indicative of ovarian cancer. In a preferred embodiment, the markers are selected from the group consisting of proteins, nucleic acids, carbohydrates, fatty acids, glycoproteins, and lipids. In another embodiment, the markers are soluble proteins.

The present invention provides methods for detecting endometrial cancer markers in a pap smear comprising the steps of: (a) obtaining a cervical pap smear, and (b) detecting the presence of said markers in said sample, wherein the presence of said markers is indicative of endometrial cancer. In a preferred embodiment, the markers are selected from the group consisting of proteins, nucleic acids, carbohydrates, fatty acids, glycoproteins, and lipids. In another embodiment, the markers are soluble proteins.

The presence of markers in a biological sample, may be used to: 1) detect the presence or absence of a gynecological cancer; 2) determine if a gynecological cancer can be or is likely to be successfully treated by an agent or combination of agents; 2) determine if a gynecological cancer is responding to treatment with an agent or combination of agents; 3) select an appropriate agent or combination of agents for treating a gynecological cancer; 4) monitor the effectiveness of an ongoing treatment; and 5) identify new treatments (either single agent or combination of agents). In particular, the biomarkers may be utilized as markers (surrogate and/or direct) to determine appropriate therapy, to monitor clinical therapy and human trials of a drug being tested for efficacy, and to develop new agents and therapeutic combinations.

Biomarkers can be detected by any means know in the art. By way of non-limiting example, biomarkers may be detected by using immunohistological, immunocytological, hybridization using immunofluorescence and/or immunoenzymatic, techniques as well as hydrometry, polarimetry, spectrophotometry (e.g., mass and NMR) and chromatography (e.g., gas liquid, high performance liquid, and thin layer).

Biomarkers of the invention are optionally recovered and purified from a biological sample by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography.

Generally, it is preferable to determine the presence of two or more of the biomarkers, more preferably, three or more of the biomarkers, most preferably, a set of the biomarkers. Thus, it is preferable to assess the presence of a panel of biomarkers.

Nucleic Acid Biomarkers

One aspect of the invention pertains to nucleic acid molecules that correspond to a biomarker. Biomarkers of the present invention include nucleic acids that encode a polypeptide corresponding to a biomarker of the invention or a portion of such a polypeptide. Biomarkers of the invention also include nucleic acid molecules sufficient for use as hybridization probes to identify nucleic acid molecules that correspond to a biomarker, including nucleic acids which encode a polypeptide corresponding to a biomarker, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid biomarker can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid biomarker can be prepared by standard synthetic techniques, e.g., using an automated DNA synthesizer.

In another preferred embodiment, a nucleic acid biomarker comprises a nucleic acid molecule that has a nucleotide sequence complementary to the nucleotide sequence of a nucleic acid corresponding to a biomarker or to the nucleotide sequence of a nucleic acid encoding a protein that corresponds to a biomarker. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid biomarker can comprise only a portion of a nucleic acid sequence, wherein the full-length nucleic acid sequence comprises a biomarker or which encodes a polypeptide corresponding to a biomarker. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid biomarker can be used to detect transcripts or genomic sequences corresponding to one or more biomarkers of the invention. The probe comprises a label group attached thereto, e.g., a radioisotope, a fluorescent compound, an enzyme, or an enzyme co-factor. Such probes can be used as part of a diagnostic test kit for identifying a biological sample, such as cells or tissues, which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid biomarkers that differ, due to degeneracy of the genetic code, from the nucleotide sequence of nucleic acids encoding a protein that corresponds to a biomarker, and thus encode the same protein.

In various embodiments, the nucleic acid biomarkers can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

The invention also includes molecular beacon nucleic acids having at least one region that is complementary to a nucleic acid biomarker, such that the molecular beacon is useful for quantitating the presence of the nucleic acid biomarker in a sample. A “molecular beacon” nucleic acid is a nucleic acid comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid are not annealed with one another, fluorescence of the fluorophore is quenched to a lesser degree. Molecular beacon nucleic acids are described, for example, in U.S. Pat. No. 5,876,930.

Protein Biomarkers and Antibodies

One aspect of the invention pertains to protein biomarkers, and biologically active portions thereof, as well as polypeptide fragments suitable for use as immunogens to raise antibodies directed against a polypeptide corresponding to a biomarker. In one embodiment, the native polypeptide corresponding to a biomarker can be isolated from a biological sample by an appropriate purification scheme using standard protein purification techniques. In another embodiment, polypeptides corresponding to a protein biomarker are produced by recombinant DNA techniques. Alternative to recombinant expression, a polypeptide corresponding to a protein biomarker can be synthesized chemically using standard peptide synthesis techniques.

Biologically active portions of a polypeptide corresponding to a protein biomarker include polypeptides comprising amino acid sequences sufficiently identical to or derived from the amino acid sequence of the protein corresponding to the biomarker, which include fewer amino acids than the full length protein, and exhibit at least one activity of the corresponding full-length protein. Typically, biologically active portions comprise a domain or motif with at least one activity of the corresponding protein. A biologically active portion of a protein biomarker can be a polypeptide that is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide.

Preferred polypeptides have the amino acid sequence listed in the one of the GenBank and NUC database records described herein. Other useful protein biomarkers are substantially identical (e.g., at least about 40%, preferably 50%, 60%, 70%, 80%, 90%, 95%, or 99%) to one of these sequences and retain the functional activity of the protein of the corresponding naturally-occurring protein yet differ in amino acid sequence due to natural allelic variation or mutagenesis.

The invention also provides chimeric or fusion proteins corresponding to a biomarker. As used herein, a “chimeric protein” or “fusion protein” comprises all or part (preferably a biologically active part) of a polypeptide corresponding to a biomarker operably linked to a heterologous polypeptide (i.e., a polypeptide other than the polypeptide corresponding to the biomarker). Within the fusion protein, the term “operably linked” is intended to indicate that the polypeptide and the heterologous polypeptide are fused in-frame to each other. The heterologous polypeptide can be fused to the amino-terminus or the carboxyl-terminus of the polypeptide.

One useful fusion protein is a GST fusion protein in which a polypeptide corresponding to a protein biomarker is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide corresponding to a biomarker of the invention can be removed and replaced with a signal sequence from another protein. For example, the gp67 secretory sequence of the baculovirus envelope protein can be used as a heterologous signal sequence (Ausubel et al., ed., Current Protocols in Molecular Biology, John Wiley & Sons, NY, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide corresponding to a biomarker of the invention is fused to sequences derived from a member of the immunoglobulin protein family.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a protein biomarker can be cloned into such an expression vector such that the fusion moiety is linked in-frame to the polypeptide.

The present invention also pertains to variants of the polypeptides corresponding to individual biomarkers of the invention. Such variants have an altered amino acid sequence can serve as probes or immunogens for antibody production. Variants can be generated by mutagenesis, e.g., discrete point mutation or truncation. An agonist can retain substantially the same, or a subset, of the biological activities of the naturally occurring form of the protein.

A polypeptide corresponding to a biomarker of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of the polypeptides, and encompasses an epitope of the protein such that an antibody raised against the peptide forms a specific immune complex with a biomarker of the invention to which the protein corresponds. Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions.

An immunogen typically is used to prepare antibodies by immunizing a suitable (i.e. immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a protein biomarker. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a protein biomarker, e.g., an epitope. A molecule which specifically binds to a given protein biomarker is a molecule which binds the polypeptide, but does not substantially bind other molecules in a sample, e.g., a biological sample, which naturally contains the polypeptide. Examples of immunologically active portions of immunoglobulin molecules include F(ab) and F(ab′).sub.2 fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen-binding site capable of immunoreacting with a particular epitope.

Polyclonal antibodies can be prepared as described above by immunizing a suitable subject with a protein biomarker as an immunogen. Preferred polyclonal antibody compositions are ones that have been selected for antibodies directed against a protein biomarker of the invention. Particularly preferred polyclonal antibody preparations are ones that contain only antibodies directed against a protein biomarker. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a protein biomarker.

The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction. Alternatively, antibodies specific for a protein biomarker can be selected or (e.g., partially purified) or purified by, e.g., affinity chromatography. For example, a recombinantly expressed and purified (or partially purified) protein biomarker is produced as described herein, and covalently or non-covalently coupled to a solid support such as, for example, a chromatography column. The column can then be used to affinity purify antibodies specific for the protein biomarker from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those of the desired protein biomarker, and preferably at most 20%, yet more preferably at most 10%, and most preferably at most 5% (by dry weight) of the sample is contaminating antibodies. A purified antibody composition means that at least 99% of the antibodies in the composition are directed against the desired protein biomarker.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a protein biomarker can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

An antibody directed against a polypeptide corresponding to a protein biomarker (e.g., a monoclonal antibody) can be used to isolate the polypeptide by standard techniques, such as affinity chromatography or immunoprecipitation. Moreover, such an antibody can be used to detect the biomarker in a biological sample in order to evaluate the level and pattern of expression of the biomarker. The antibodies can also be used diagnostically to monitor protein levels in biological sample (e.g., tissues or body fluids). Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, .beta.-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include .sup.125I, .sup.131I, .sup.35S or .sup.3H.

Any of the antibodies mentioned above can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

The invention also provides assays and kits for the detection of the presence or absence of a biomarker in a biological sample, and instructions for use.

An exemplary method for detecting the presence or absence of a polypeptide or nucleic acid corresponding to a biomarker of the invention in a biological sample involves obtaining a biological sample (e.g. a cervical pap smear) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a polypeptide corresponding to a biomarker include enzyme linked immunosorbent assays (ELISAs), Western blots, immunoprecipitations and immunofluorescence. In vitro techniques for detection of genomic DNA include Southern hybridizations. Furthermore, in vivo techniques for detection of a polypeptide corresponding to a biomarker include introducing into a subject a labeled antibody directed against the polypeptide. For example, the antibody can be labeled with a radioactive biomarker whose presence and location in a subject can be detected by standard imaging techniques.

A general principle of such diagnostic and prognostic assays involves preparing a sample or reaction mixture that may contain a biomarker, and a probe, under appropriate conditions and for a time sufficient to allow the biomarker and probe to interact and bind, thus forming a complex that can be removed and/or detected in the reaction mixture. These assays can be conducted in a variety of ways.

For example, one method to conduct such an assay would involve anchoring the biomarker or probe onto a solid phase support, also referred to as a substrate, and detecting target biomarker/probe complexes anchored on the solid phase at the end of the reaction. In one embodiment of such a method, a sample from a subject, which is to be assayed for presence and/or concentration of biomarker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, biomarker or probe molecules that are immobilized through conjugation of biotin and streptavidin. Such biotinylated assay components can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the biomarker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above-mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of biomarker/probe complexes anchored to the solid phase can be accomplished in a number of methods outlined herein.

For example, the probe, when it is the unanchored assay component, can be labeled for the purpose of detection and readout of the assay, either directly or indirectly, with detectable labels discussed herein and which are well-known to one skilled in the art.

It is also possible to directly detect biomarker/probe complex formation without further manipulation or labeling of either component (biomarker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

For example, determination of the ability of a probe to recognize a biomarker can be accomplished without labeling either assay component (probe or biomarker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In a preferred embodiment, analogous diagnostic and prognostic assays can be conducted with biomarker and probe as solutes in a liquid phase. In such an assay, the complexed biomarker and probe are separated from uncomplexed components by any of a number of standard techniques, including but not limited to: differential centrifugation, chromatography, electrophoresis and immunoprecipitation. In differential centrifugation, biomarker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the biomarker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl Oct. 10, 1997;699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of mRNA corresponding to the biomarker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from ovarian cells (see, e.g., Ausubel et al, ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a biomarker of the present invention. Hybridization of an mRNA with the probe indicates that the biomarker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the biomarkers of the present invention.

An alternative method for determining the level of mRNA corresponding to a biomarker in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the ovarian cells prior to detection. In such methods, a cell or tissue sample is prepared/processed using known histological methods. The sample is then immobilized on a support, typically a glass slide, and then contacted with a probe that can hybridize to mRNA that encodes the biomarker.

As an alternative to making determinations based on the absolute expression level of the biomarker, determinations may be based on the normalized expression level of the biomarker. Expression levels are normalized by correcting the absolute expression level of a biomarker by comparing its expression to the expression of a gene that is not a biomarker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-ovarian cancer sample, or between samples from different sources.

Alternatively, the expression level can be provided as a relative expression level. To determine a relative expression level of a biomarker, the level of expression of the biomarker is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the biomarker. The expression level of the biomarker determined for the test sample (absolute level of expression) is then divided by the mean expression value obtained for that biomarker. This provides a relative expression level.

In another embodiment of the present invention, a polypeptide corresponding to a biomarker is detected. A preferred agent for detecting a polypeptide of the invention is an antibody capable of binding to a polypeptide corresponding to a biomarker of the invention, preferably an antibody with a detectable label. Antibodies can be polyclonal, or more preferably, monoclonal. An intact antibody, or a fragment thereof (e.g., Fab or F(ab′).sub.2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether ovarian cells express a biomarker of the present invention.

In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from ovarian cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

The invention also encompasses kits for detecting the presence of a polypeptide or nucleic acid corresponding to a biomarker of the invention in a biological sample (e.g. an ovary-associated body fluid such as a urine sample). Such kits can be used to determine if a subject is suffering from or is at increased risk of developing ovarian cancer. For example, the kit can comprise a labeled compound or agent capable of detecting a polypeptide or an mRNA encoding a polypeptide corresponding to a biomarker of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: (1) a first antibody (e.g., attached to a solid support) which binds to a polypeptide corresponding to a biomarker of the invention; and, optionally, (2) a second, different antibody which binds to either the polypeptide or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a polypeptide corresponding to a biomarker of the invention or (2) a pair of primers useful for amplifying a nucleic acid molecule corresponding to a biomarker of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples that can be assayed and compared to the test sample. Each component of the kit can be enclosed within an individual container and all of the various containers can be within a single package, along with instructions for interpreting the results of the assays performed using the kit.

The scientific literature is replete with post-translational modified proteins as biomarkers. Most notable are the glycosylation patterns of cell surface aspargine-N-linked glycoconjugates including but not limited to classes of biological compounds known as glycoproteins, glycolipids and gangliosides. A separate but related class of glycoconjugates includes what is commonly referred to by those versed in the art as the mucin-type glycoproteins, are characterized by 0-linked oligosaccahride structures. Variations in oligosaccahride structure of glycoconjugates in pre-cancerous and cancerous state have been well-documented in the scientific literature (examples; Lewis X antigens, CA125, CA 15-3 and CA 19-9 determinants). Antibodies have been produced to these classes of biomarkers assays developed and ELISA kits produced to quantification in serum, plasma and other body fluids. Cancer-associated glycoconjugates generally vary from their normal counterparts in the proportion terminal sialic acid (N-acetyl neurominic acid) residues typically linked in a 6-O glycosidic bond to the penultimate galactose residue of the N-linked class of glycoproteins.

The presence of abnormal levels of novel bioactive lipids have also been associated with pre-neoplastic and malignant conditions. Of note as an example of this class of biomarker is the documented presence of lysophosphatidic acid (LPA) in as early stage indicator of ovarian cancer (Gordon Mills and Michael Skinner). An independent study (JAMA 1998) demonstrated 90% accuracy in detecting Stage I ovarian cancer utilizing a lipid-based approach and was 100% accurate in detecting Stage II, III, and IV ovarian cancer from blood. As these classes of bioactive lipids have been shown to indicative of ovarian cancer, it may be suggested they could be presented and detected in the extracellular milieu collected during the process of Pap testing and subsequently collected into a liquid medium.

In addition to the demonstrated post-translationally modified proteins and peptides as biomarkers, hypermethylation of specific guanosine-containing regions of DNA have been demonstrated to be early indicators of the malignant condition (James Herman at JHU). Hypermethylated DNA may also be collected from Pap specimens as early indicators of ovarian and/or endometrial cancer. Hypermethylated DNA is not typically detected immunologically, but can be detected by methylation-specific PCR where hypermethylated CpG islands are converted to uracil containing regions by a bisulfite treatment which is followed by PCR using specifically designed primers.

Lipids, gycolipids, glycoproteins, other non-limiting glcoconjugates, hypermethylated and acetylated DNA should definitely be included in as potential biomarkers of early ovarian and endometrial cancer detection in this application.

Equivalents

Those skilled in the art will recognize, or be able to ascertain using no more than routine experimentation, many equivalents to the specific embodiments of the invention described herein. Such equivalents are intended to be encompassed by the following claims.

1. A method for detecting biomarkers in a biological sample, comprising the steps of:
(a) obtaining a cervical pap specimen and
(b) detecting the presence of biomarkers in said sample;
wherein the presence of said biomarkers is indicative the presence of a non-cervical cancer.

2. The method of claim 1, wherein said cancer is ovarian or endometrial cancer.

3. The method of claim 1, wherein said biomarkers are freely soluble proteins.

4. The method of claim 1, wherein said biomarkers are membrane associated.

5. The method of claim 1, wherein said biomarkers are selected from the group consisting of proteins, nucleic acids, carbohydrates, fatty acids, glycoproteins, and lipids.

6. The method of claim 1, wherein said biological sample is from a cervical scraping collected for liquid-based cytology

7. A method for detecting ovarian cancer biomarkers in a pap smear, comprising the steps of:
(a) obtaining a cervical pap specimen and
(b) detecting the presence of said biomarkers in said sample;
wherein the presence of said biomarkers is indicative of ovarian cancer.

8. The method of claim 7, wherein said biomarkers are freely soluble proteins.

9. The method of claim 7, wherein said biomarkers are membrane associated.

10. The method of claim 7, wherein said biomarkers are selected from the group consisting of proteins, nucleic acids, carbohydrates, fatty acids, glycoproteins, and lipids.

11. The method of claim 7, wherein said biological sample is from a cervical scraping collected for liquid-based cytology

12. A method for detecting endometrial cancer biomarkers in a pap smear, comprising the steps of:
(a) obtaining a cervical pap specimin and
(b) detecting the presence of said biomarkers in said sample;
wherein the presence of said biomarkers is indicative of endometrial cancer.

13. The method of claim 12, wherein said biomarkers are freely soluble proteins.

14. The method of claim 12, wherein said biomarkers are membrane associated.

15. The method of claim 12, wherein said biomarkers are selected from the group consisting of proteins, nucleic acids, carbohydrates, fatty acids, glycoproteins, and lipids.

16. The method of claim 1, wherein said biological sample is from a cervical scraping collected for liquid-based cytology
